(19) United States
(12) Patent Application Publication
(10) Pub. No.: US 2026/0314654 A1
(43) Pub. Date: Oct. 15, 2026

(54) METHYL CELLULOSE AS A HUMECTANT IN POUCH SUBSTRATES

(71) Applicant: Philip Morris Products S.A., Neuchatel (CH)

(72) Inventor: Kent Albin NIELSEN, Brande (DK)

(73) Assignee: Philip Morris Products S.A., Neuchatel (CH)

(21) Appl. No.: 19/482,363

(22) PCT Filed: May 10, 2024
(86) PCT No.: PCT/EP2024/062931

§ 371 Date: Nov. 7, 2025

(30) Foreign Application Priority Data
May 12, 2023 (EP) 23173035.9

(51) Int. Cl. A24B 15/40; A24B 13/00

(52) U.S. Cl. CPC A24B 15/403; A24B 13/00

(57) ABSTRACT

Methyl cellulose as a humectant in a pouch substrate is provided, the methyl cellulose having a viscosity at 20° C. of about 200 cP to about 10000 cP at 2% w/v concentration, and the methyl cellulose being added in an amount of about 0.5 wt %-6 wt % based on the total weight of the pouch substrate. A pouch for oral use including a pouch substrate is also provided, the pouch substrate including one or more carriers, one or more active ingredients, and methyl cellulose having a viscosity at 20° C. of about 200 cP to about 10000 cP at 2% w/v concentration, the methyl cellulose being added in an amount of about 0.5 wt %-6 wt % based on the total weight of the pouch substrate.

### FIELD OF THE INVENTION

The present invention relates to a use of methyl cellulose as a humectant in a pouch substrate. The invention also relates to a pouch for oral use comprising a pouch substrate, wherein said pouch substrate comprises one or more carriers, one or more active ingredients, and methyl cellulose having a viscosity at 20° C. of 200 to 10000 cP at 2% w/v concentration.

### BACKGROUND OF THE INVENTION

A popular product for smoke-free oral delivery of active ingredients such as nicotine is a pouch. A pouch is a container formed of a water-permeable material enclosing a cavity. The pouch is filled with a pouch substrate and then sealed. The pouch substrate can be dry, semi-moist, or moist. Pouch substrates often contain alginate or other similar polysaccharides as humectants. Alginate improves the processability of the pouch substrate and retains moisture. Oral compositions comprising polysaccharides (e.g. acacia gum, xanthan gum, alginate, carrageen, etc.) which are used in pouches are described in US 2021/0169890 A1.

A problem associated with the use of alginate or other similar polysaccharides in pouch substrates is a decreased release rate of the active ingredient (e.g. nicotine) and flavoring agent(s) contained in the pouch substrate. This results in an inefficient delivery of the active ingredient(s) and flavoring agent(s).

There is thus a need for humectants which not only improve the processability (powder flowability, reduced dust formation, etc.) of a pouch substrate but also allow a more complete and faster release of the active ingredient(s) and flavoring agent(s) contained in the pouch substrate.

### OBJECT OF THE INVENTION

An object of the present invention is to provide a humectant which improves the processability of a pouch substrate, retains the moisture in the pouch substrate and allows a more complete and faster release of the active ingredient(s) and flavoring agent(s) contained in the pouch substrate compared to known humectants (e.g. alginate). Another object of the invention is to provide a pouch for oral use which exhibits improved delivery of the active ingredient(s) and flavoring agent(s) contained therein, and as a result provides superior user experience.

The use and the pouch for oral use described below satisfy the above aims.

### SUMMARY OF THE INVENTION

In one aspect, the invention relates to the use of methyl cellulose as a humectant in a pouch substrate.

In another aspect, the invention relates to a pouch for oral use comprising a pouch substrate, wherein said pouch substrate comprises one or more carriers, one or more active ingredients and methyl cellulose having a viscosity at 20° C. of about 200 to about 10000 cP at 2% w/v concentration.

In a preferred embodiment of each of the above aspects, the pouch substrate further comprises one or more carriers and one or more active ingredients.

In a further preferred embodiment of each of the above aspects, the active ingredient is nicotine.

In a further preferred embodiment of each of the above aspects, the methyl cellulose has a viscosity at 20° C. of about 200 to about 10000 cP at 2% w/v concentration.

In a preferred embodiment of each of the above aspects, the methyl cellulose contains about 27.5-31.5 wt % of methoxy groups.

In a further preferred embodiment of each of the above aspects, the methyl cellulose has a viscosity at 20° C. of about 300 to about 5000 cP at 2% w/v concentration.

In a preferred embodiment of each of the above aspects, the methyl cellulose is added in an amount of about 0.5 wt %-10 wt % based on the total weight of the pouch substrate.

In a further preferred embodiment of each of the above aspects, the pouch substrate further comprises one or more flavoring agents.

### DESCRIPTION OF THE FIGURE

FIG. 1: Nicotine release of the nicotine contained in the pouch substrates of Examples 1 and 2 and Comparative Example 1 as a function of time.

### DETAILED DESCRIPTION OF THE INVENTION

The present invention is based on the finding that the use of methyl cellulose (MC) as a humectant in a pouch substrate not only improves the processability and moisture retention of the pouch substrate but also improves the release of the active ingredient(s) and flavoring agent(s) contained in the pouch substrate. Thus, a pouch comprising methyl cellulose as a humectant has an improved potency and flavor intensity and provides a superior user experience.

### Definitions and Terminology

All technical and scientific terms used hereafter carry the commonly understood meaning of the word unless defined otherwise.

Unless defined otherwise, any feature(s) within any aspect or embodiment of the invention may be combined with any feature(s) within any other aspect or embodiment of the invention, and the skilled person understands such combination as being encompassed in the original disclosure of the present application. This also applies to the possibility of combination of elements from lists of species within definitions of different broader terms with one another, said possibilities of individualized combination being expressly intended and within the direct and unambiguous disclosure of the present application. This also applies in particular, but not exclusively, to endpoints of ranges disclosed herein. For instance, if a given substance is disclosed as existing in a composition in a concentration range of X-Y % or A-B %, the present application is to be understood as explicitly disclosing not only the ranges X-Y % and A-B %, but also the ranges X-B %, A-Y % and, in as far as numerically possible, Y-A % and B-X %. Each of these ranges, and range combinations, are contemplated, and are to be understood as being directly and unambiguously disclosed in the present application.

Similarly, it is also to be understood that section headings as used throughout the present application are merely for organizational purposes, and do not confer exclusive character to the disclosure pertaining to aspects or embodiments of the invention mentioned in a particular section heading. Section headings thus do not exclude the combination of the disclosure within any one section heading to a corresponding or analogous context provided under another section heading. Indeed, such intra-section disclosures are explicitly within the disclosure of the present application.

As used herein, each of the terms “comprising’, “having” and “containing”, including grammatical variants thereof, are meant in a non-exhaustive sense to mean “including”, but not necessarily “composed of”, and does not exclude elements in addition to those explicitly recited as being present. As used herein, then, the terms “comprising”, “having” and “containing”, and grammatical variants thereof, indicate that components other than those explicitly recited may, but need not, be present. As such these terms include as a limiting case embodiments in which no other elements than those recited are present, e.g. in the commonly accepted sense of “consisting of”.

As used herein, the phrase “consisting of”, and grammatically related variants thereof, means that no other elements are present than those recited. In standing with the above definition of “comprising” (and grammatically and semantically related terms), the term “consisting of” therefore denotes a limiting scenario within the meaning of “comprising”.

Unless stated otherwise, percentages indicating the extent to which a particular ingredient is comprised in a mixture or composition refer to weight/weight (w/w) percentages. Similarly, unless stated otherwise, ratios specified herein are weight ratios.

Unless stated otherwise, the designation of a range in the present application using a hyphen (“-”) separating two bracketing values X and Y, or two bracketing ratios, is to be understood as meaning and disclosing the specified range in which both endpoint values X and Y are included. The same applies to a range expressed as “from X to Y”. Accordingly, the expressions of ranges as “X-Y”, “of X to Y”, “from X to Y”, “of X-Y” and “from X-Y” are to be understood equivalently as meaning and disclosing a range encompassing the end value X, all values between X and Y, as well as the end value Y. Unless stated otherwise, the designation of a range as “not more than X” is to be understood as meaning and disclosing a range whose upper limit is X and which includes the value X. Unless stated otherwise, the designation of a range as “more than X and up to Y” is to be understood as meaning and disclosing a range whose lower limit does not include the value X but includes any value higher than X up and including an upper limit Y. As used herein “any value higher than X” may encompass the value which is 1.0% higher than X.

As used herein the term “about” when referring to a particular value, e.g. an endpoint or endpoints of a range, encompasses and discloses, in addition to the specifically recited value itself, a certain variation around that specifically recited value. Such a variation may for example arise from normal measurement variability, e.g. in the weighing or apportioning of various substances by methods known to the skilled person. The term “about” shall be understood as encompassing and disclosing a range of variability above and below an indicated specific value, said percentage values being relative to the specific recited value itself, as follows: The term “about” may encompass and disclose variability of ±5.0%. The term “about” may encompass and disclose variability of ±4.5%. The term “about” may encompass and disclose variability of ±4.0%. The term “about” may encompass and disclose variability of ±3.5%. The term “about” may encompass and disclose variability of ±3.0%. The term “about” may encompass and disclose variability of ±2.5%. The term “about” may encompass and disclose variability of ±2.0%. The term “about” may encompass and disclose variability of ±1.5%. The term “about” may encompass and disclose variability of ±1.0%. The term “about” may encompass and disclose variability of ±0.5%. The term “about”, in reference to the particular recited value, may encompass and disclose that exact particular value itself, irrespective of any explicit mention that this exact particular value is included; even in the absence of an explicit indication that the term “about” includes the particular exact recited value, this exact particular value is still included in the range of variation created by the term “about”, and is therefore disclosed in the present application. Unless stated otherwise, where the term “about” is recited before the first endpoint of a numerical range, but not before the second endpoint of that range, this term, and the variability it implies in scope and disclosure, refers to both the first endpoint of the range and the second endpoint of the range. For instance, a recited range of “about X to Y” should be read as “about X to about Y”. The same applies for a recited range of ratios. For instance, a recited range of weight ratios of “about X:Y-A:B” should be read as a weight ratio of “(about X):(about Y)-(about A):(about B)”.

As used herein, the term “a combination thereof” denotes any combination thereof, in any proportion. That is, the term encompasses any combination of the listed features (e.g. a combination of any two listed features, any three listed features, etc.) in any relative amounts.

As used herein, the term “humectant” denotes a substance which is hygroscopic (water-absorbing) and retains the moisture of the pouch substrate.

As used herein, the term “pouch substrate” denotes a composition which is suitable for use in a pouch for oral use.

As used herein, the term “for oral use” denotes a product which is adapted for use in a user's mouth. During use, saliva in the mouth of the user causes one or more of the components of the product (e.g. pouch substrate) to pass into the mouth of the user.

As used herein, the term “dry” in the context of the pouch substrate means a water content of not more than 12 wt %.

As used herein, the term “semi-moist” in the context of the pouch substrate means a water content of more than 12 wt % and not more than 20 wt %.

As used herein, the term “moist” in the context of the pouch substrate means a water content of more than 20 wt % and not more than 50 wt %.

As used herein, the term “water content” refers to gravimetric water content expressed by mass (weight) as follows u=m_{w}/mₛ where m_{w} is the mass of water and mₛ is the mass of the solids. The water content can be determined by the Karl Fischer method, specifically according to ISO 6488:2021—Tobacco and tobacco products—Determination of water content—Karl Fischer method.

As used herein, the term “pouch” denotes a container formed of a water-permeable material enclosing a cavity. The pouch is adapted for oral use. The water-permeable material can be a fibrous material such a woven or non-woven fabric. The non-woven fabric can be made of polyester fibers, viscose, sisal, wool fibers or a combination thereof. The water-permeable material is not water-soluble. In a preferred embodiment, the fibrous material is a non-woven fabric made of a combination of polyester and viscose. Preferably, the weight ratio of polyester to viscose is about 1:1 to 10:1, more preferably about 2:1 to 5:1, most preferably about 4:1. In another preferred embodiment, the water-permeable material is a non-woven fabric which is laminated with a layer of thermoplastic polymer (e.g. thermoplastic polyurethane).

As used herein, the term “water-permeable” means that water can pass bidirectionally through the material of the pouch, i.e. from the oral cavity into the interior of the pouch, as well as from inside the pouch (back) into the oral cavity.

As used herein, the term “carrier” denotes a substance or a group of substances whose content is 50 wt % or more of the pouch substrate. Non-limiting examples of carriers include water-insoluble fibers, powdered cellulose and sugar alcohols. A sugar alcohol may for example be selected from the group consisting of xylitol, maltitol, mannitol, erythritol, isomalt, sorbitol, lactitol, and a combination thereof.

As used herein, the term “water-insoluble fiber” denotes a substance which is water-insoluble and is in fiber form. Non-limiting examples thereof may be chosen from the group consisting of cellulose fiber (e.g. powdered cellulose), wheat fiber, pea fiber, rice fiber, maize fiber, oat fiber, tomato fiber, barley fiber, rye fiber, sugar beet fiber, buckwheat fiber, potato fiber, apple fiber, cocoa fiber, bran fiber, bamboo fiber, and a combination thereof.

As used herein, the term “water-insoluble” refers to water solubility of less than 0.1 gram of water-soluble composition or substance per 100 mL of water, measured at 25° C., atmospheric pressure and pH of 7.

As used herein, the term “powder form” refers to a composition in the form of a powder, i.e. a particulate material having a relatively small average particle size, for example, 1-1500 μm.

As used herein, the term “fiber form” refers to a composition in the form of fibers, i.e. particles which are significantly longer than they are wide. The term “fiber form” also encompasses all products referred to in the field as dietary fibers.

As used herein, the term “active ingredient” denotes a substance that has a modulating, e.g. a stimulating effect on the nervous system of a user. In a preferred embodiment, the active ingredient is selected from the group consisting of nicotine, caffeine, anatabine, one or more cannabinoids and a combination thereof in any proportion.

As used herein, the term “nicotine” denotes a nicotine source. The nicotine source can be any nicotinic compound. Such nicotinic compound includes nicotine in free base form (i.e. nicotine in a non-protonated form), salt form, as a complex, or as a solvate. Example of a nicotine complex is a resin complex of nicotine, where nicotine is bound in an ion exchange resin, such as nicotine polacrilex. Examples of nicotine salts are benzoate, hydrochloride, dihydrochloride, monotartrate, bitartrate, sulfate, salicylate, citrate, stearate, glutarate, aspartate, palmitate, and lactate. Further possible forms of nicotine are discussed below. A combination thereof in any proportion is also possible and disclosed in the context of the present invention.

As used herein “nicotine content” denotes the content of the nicotine source calculated as free base nicotine in weight/weight (w/w) percentage, also referred to herein as “wt %”.

As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). A combination thereof in any proportion is also possible and disclosed in the context of the present invention.

As used herein, the term “flavoring agent” is any flavorful or aromatic substance capable of altering the sensory characteristics of a compound or a composition. Examples of sensory characteristics that can be modified by the flavoring agent include taste (e.g. sweet, salty, sour, bitter, etc.), mouthfeel, moistness, coolness/heat, and/or fragrance/aroma. Flavoring agents may be natural or synthetic, and the character of the flavors imparted thereby may be described, without limitation, as fresh, sweet, salty, umami, herbal, confectionary, floral, fruity, or spicy. Specific types of flavors include, but are not limited to, vanilla, coffee, chocolate/cocoa, cream, mint, spearmint, menthol, peppermint, wasabi, azuki, ginseng, kelp, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, and any combination thereof in any proportion. Flavoring agents also include components that are considered moistening, cooling or smoothening agents, such as eucalyptus. The flavoring agent can mask the flavor of other components in a composition (e.g. bitterness). For example, a sweetener can be used as a bitterness masking agent. The flavoring agent may comprise at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. Examples of volatile flavor components include alcohols, aldehydes, aromatic hydrocarbons, ketones, esters, terpenes, terpenoids, trigeminal sensates, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-famesene, nerolidol, thujone, myrcene, geraniol, nerol, citronellol, linalool, and eucalyptol. A combination thereof in any proportion is also possible and disclosed in the context of the present invention.

### Use of Methyl Cellulose as a Humectant in a Pouch Substrate

The present invention provides a use of methyl cellulose as a humectant in a pouch substrate. It has been surprisingly found that methyl cellulose is superior to conventional humectants (e.g. alginates) since it provides superior release of active ingredient(s) and flavoring agent(s) contained in a pouch substrate.

The pouch substrate can be dry, semi-moist, or moist. In a preferred embodiment, the pouch substrate is moist.

In a preferred embodiment, the pouch substrate further comprises one or more carriers and one or more active ingredients.

In a further preferred embodiment, the pouch substrate has a water content of about 13 wt %-35 wt %, preferably about 15 wt %-30 wt %, more preferably about 17 wt %-25 wt %.

In a further preferred embodiment, the carrier comprises a water-insoluble fiber and/or a sugar alcohol. The content of the carrier in the pouch substrate can be at least about 50 wt %, preferably about 50 wt %-90 wt %, more preferably about 60 wt %-85 wt %, and most preferably about 70 wt %-80 wt %.

In a preferred embodiment, in combination with any of the above or below embodiments, the sugar alcohol is selected from the group consisting of xylitol, maltitol, mannitol, erythritol, isomalt, sorbitol, lactitol, and a combination thereof. Preferably, the sugar alcohol is a combination of xylitol and erythritol.

In a further preferred embodiment, in combination with any of the above or below embodiments, the water-insoluble fiber is selected from the group consisting of cellulose fiber (e.g. powdered cellulose), wheat fiber, pea fiber, rice fiber, maize fiber, oat fiber, tomato fiber, barley fiber, rye fiber, sugar beet fiber, buckwheat fiber, potato fiber, apple fiber, cocoa fiber, bran fiber, bamboo fiber, and a combination thereof.

In a preferred embodiment, the active ingredient is nicotine. Nicotine in any form can be used. The nicotine can be formulated in a premix of a liquid suspension prior to its addition to the pouch substrate.

In a preferred embodiment, the nicotine is a nicotine salt or a resin complex of nicotine, where nicotine is bound in an ion exchange resin (i.e. nicotine/ion exchange resin complex). In another preferred embodiment, the nicotine salt is nicotine bitartrate, glutarate, aspartate, palmitate, or lactate, most preferably nicotine bitartrate. A combination thereof in any proportion is also possible and disclosed in the context of the present invention.

In a further preferred embodiment, the nicotine can be a liquid form of nicotine (e.g. nicotine free base) which is encapsulated (e. g. by means of cyclo-dextrins).

In a preferred embodiment, the nicotine is selected from the group consisting of nicotine bitartrate, nicotine polacrilex, nicotine betadex, nicotine o-salicylate, nicotine gentisate, nicotine gallate, nicotine phthalate, nicotine isophthalate, nicotine-acesulfame H, nicotine benzoate, nicotine fumarate, nicotine R-mandelate, nicotine hydrochlorate, nicotine L-lactate, nicotine levulinate, nicotine phosphorate, nicotine ascorbate, nicotine ferulate, nicotine vanilate, nicotine picolinate, nicotine cumate, nicotine nicotinate, nicotine pyruvate, and nicotine gamma-resorcylate. A combination thereof in any proportion is also possible and disclosed in the context of the present invention.

In a preferred embodiment, the active ingredient is nicotine and the content thereof in the pouch substrate is about 0.5-20 mg, preferably about 0.5-15 mg, most preferably about 0.5-10 mg.

In a preferred embodiment, the methyl cellulose has a viscosity at 20° C. of about 200 to about 10000 cP at 2% w/v concentration. More preferably, the methyl cellulose has a viscosity at 20° C. of about 300 to about 5000 cP at 2% w/v concentration. The viscosity is measured according to the method described in Ph. Eur. 11.0, pages 3379-3380.

In a preferred embodiment, the methyl cellulose has a viscosity at 20° C. of about 300 to about 500 cP at 2% w/v concentration. An example of such methyl cellulose is Methocel™ 4AC by DuPont.

In another preferred embodiment, the methyl cellulose has a viscosity at 20° C. of about 2000 to about 5000 cP at 2% w/v concentration, preferably a viscosity at 20° C. of 2500 to 5000 cP at 2% w/v concentration. An example of such methyl cellulose is Methocel™ 4AM by DuPont.

In a further preferred embodiment, the methyl cellulose contains about 27.5-31.5 wt % of methoxy groups. The methoxy group content can be determined by NMR spectroscopy.

In a further preferred embodiment, the methyl cellulose is added in an amount of about 0.5 wt %-10 wt % based on the total weight of the pouch substrate. More preferably, the methyl cellulose is added in an amount of about 1 wt %-8 wt % based on the total weight of the pouch substrate, even more preferably about 1.2 wt %-6 wt % based on the total weight of the pouch substrate, and most preferably about 1.3 wt %-5 wt % based on the total weight of the pouch substrate.

In a preferred embodiment, the pouch substrate further comprises one or more flavoring agents.

In a further preferred embodiment, the pouch substrate comprises methyl cellulose having a viscosity at 20° C. of about 300 to about 5000 cP at 2% w/v concentration, one or more carriers, one or more active ingredients and one or more flavoring agents, wherein the carrier is a water-insoluble fiber and the active ingredient is nicotine.

All preferred embodiments described above can be combined with each other, and such combinations are disclosed in the context of the present invention.

### Pouch for Oral Use

The present invention also provides a pouch for oral use comprising a pouch substrate, wherein said pouch substrate comprises one or more carriers, one or more active ingredients and methyl cellulose having a viscosity at 20° C. of about 200 to about 10000 cP at 2% w/v concentration. The viscosity is measured according to the method described in Ph. Eur. 11.0, pages 3379-3380.

The pouch substrate can be dry, semi-moist, or moist. In a preferred embodiment, the pouch substrate is moist.

In a further preferred embodiment, the pouch substrate has a water content of about 13 wt %-35 wt %, preferably about 15 wt %-30 wt %, more preferably about 17 wt %-25 wt %.

In a further preferred embodiment, the carrier comprises a water-insoluble fiber and/or a sugar alcohol. The content of the carrier in the pouch substrate can be at least about 50 wt %, preferably about 50 wt %-90 wt %, more preferably about 60 wt %-85 wt %, and most preferably about 70 wt %-80 wt %.

In a preferred embodiment, in combination with any of the above or below embodiments, the sugar alcohol is selected from the group consisting of xylitol, maltitol, mannitol, erythritol, isomalt, sorbitol, lactitol, and a combination thereof.

In a further preferred embodiment, in combination with any of the above or below embodiments, the water-insoluble fiber is selected from the group consisting of cellulose fiber (e.g. powdered cellulose), wheat fiber, pea fiber, rice fiber, maize fiber, oat fiber, tomato fiber, barley fiber, rye fiber, sugar beet fiber, buckwheat fiber, potato fiber, apple fiber, cocoa fiber, bran fiber, bamboo fiber, and a combination thereof.

In a preferred embodiment, the active ingredient is nicotine. Nicotine in any form can be used. The nicotine can be formulated in a premix of a liquid suspension prior to its addition to the pouch substrate.

In a preferred embodiment, the nicotine is a nicotine salt or a resin complex of nicotine, where nicotine is bound in an ion exchange resin (i.e. nicotine/ion exchange resin complex). In another preferred embodiment, the nicotine salt is nicotine bitartrate, glutarate, aspartate, palmitate, or lactate, most preferably nicotine bitartrate.

In a further preferred embodiment, the nicotine can be a liquid form of nicotine (e.g. nicotine free base) which is encapsulated (e. g. by means of cyclo-dextrins).

In a preferred embodiment, the nicotine is selected from the group consisting of nicotine bitartrate, nicotine polacrilex, nicotine betadex, nicotine o-salicylate, nicotine gentisate, nicotine gallate, nicotine phthalate, nicotine isophthalate, nicotine-acesulfame H, nicotine benzoate, nicotine fumarate, nicotine R-mandelate, nicotine hydrochlorate, nicotine L-lactate, nicotine levulinate, nicotine phosphorate, nicotine ascorbate, nicotine ferulate, nicotine vanilate, nicotine picolinate, nicotine cumate, nicotine nicotinate, nicotine pyruvate, and nicotine gamma-resorcylate. A combination thereof in any proportion is also possible and disclosed in the context of the present invention.

In a preferred embodiment, the active ingredient is nicotine and the content thereof in the pouch substrate is about 0.5-20 mg, preferably about 0.5-15 mg, most preferably about 0.5-10 mg.

In a preferred embodiment, the methyl cellulose has a viscosity at 20° C. of about 300 to about 5000 cP at 2% w/v concentration.

In a preferred embodiment, the methyl cellulose has a viscosity at 20° C. of about 300 to about 500 cP at 2% w/v concentration. In another preferred embodiment, the methyl cellulose has a viscosity at 20° C. of about 2000 to about 5000 cP at 2% w/v concentration, preferably a viscosity at 20° C. of about 2500 to about 5000 cP at 2% w/v concentration.

In a further preferred embodiment, the methyl cellulose contains about 27.5-31.5 wt % of methoxy groups. The methoxy group content can be determined by NMR spectroscopy.

In a further preferred embodiment, the methyl cellulose is added in an amount of about 0.5 wt %-10 wt % based on the total weight of the pouch substrate. More preferably, the methyl cellulose is added in an amount of about 1 wt %-8 wt % based on the total weight of the pouch substrate, even more preferably about 1.2 wt %-6 wt % based on the total weight of the pouch substrate, and most preferably about 1.3 wt %-5 wt % based on the total weight of the pouch substrate.

In a preferred embodiment, the pouch substrate further comprises one or more flavoring agents.

In a further preferred embodiment, the pouch substrate comprises methyl cellulose having a viscosity at 20° C. of about 300 to about 5000 cP at 2% w/v concentration, one or more carriers, one or more active ingredients and one or more flavoring agents, wherein the carrier is a water-insoluble fiber and the active ingredient is nicotine.

All preferred embodiments described above can be combined with each other, and such combinations are disclosed in the context of the present invention.

The pouch substrates described herein may optionally contain one or more pH adjusting agents, preservatives, flavor enhancers and sweeteners. Examples of pH adjusting agents include sodium orthophosphate, potassium orthophosphate, calcium orthophosphate, sodium diphosphate, potassium diphosphate, calcium diphosphate, pentasodium triphosphate, pentapotassium triphosphate, sodium polyphosphate, potassium polyphosphate, carbonic acid, sodium carbonate, sodium bicarbonate, potasium carbonate, calcium carbonate, magnesium carbonate, and a combination thereof. Examples of preservatives include sorbic acid and its salts (e.g. potassium-sorbate), benzoic acid and its salts, sulfur dioxide salts, nitrate salts, nitrite salts, acetic acid and its salts, lactic acid and its salts, malic acid and its salts, and a combination thereof. An example of a flavor enhancer is sodium chloride. Examples of sweeteners include sugar sweeteners such as sucrose, dextrose, maltose, and lactose and high-intensity sweeteners such as sucralose, aspartame, salts of acesulfame (e.g. acesulfame potassium), alitame, saccharin and its salts, cyclamic acid and its salts, glycyrrhizin, dihydrochalcones, thaumatin, monellin, stevioside, and a combination thereof.

### Examples

Pouch substrates having the compositions given in Table 1 were prepared and filled into pouches according to the method of Example 3A of WO 2020/244721 A1. The nicotine direct premix was prepared according to the method of Example 2B of WO 2020/244721 A1.

TABLE 1

| | Exam- | Exam- | Comparative |
|---|---|---|---|
| | ple 1 | ple 2 | Example 1 |
| Erythritol (Zerose ™ standard | 20.4 | 20.4 | 20.4 |
| granular available from | | | |
| Cargill) (wt %) | | | |
| Nicotine direct premix: 13.2 | 12.1 | 12.1 | 12.1 |
| wt % nicotine (wt %) | | | |
| Sodium carbonate (wt %) | 3.0 | 3.0 | 3.0 |
| Sodium alginate (wt %) | — | — | 1.5 |
| Methocel ™ A4M | 1.5 | — | — |
| Methocel ™ A4C | — | 1.5 | — |
| Wheat fiber (Vitacel WF 600 | 30.0 | 30.0 | 30.0 |
| R available from JRS) (wt %) | | | |
| Purified water (wt %) | 25.0 | 25.0 | 25.0 |
| Mint Flavor (wt %) | 1.0 | 1.0 | 1.0 |
| Xylitol (Xylisorb XTAB 400 | 5.0 | 5.0 | 5.0 |
| available from Roquette) | | | |
| (wt %) | | | |
| Silicon dioxide | 2.0 | 2.0 | 2.0 |

### Nicotine Release Test:

Nicotine release from the pouches of Examples 1 and 2 and Comparative Example 1 as a function of time was measured according to the following method:

A chewing buffer was prepared by dissolving 2.72 g of potassium dihydrogen phosphate in 950 ml purified water and the pH of the solution was adjusted to 7.4 with 2 N KOH solution. Purified water was added to obtain a final buffer amount of 1000 ml.

10 liter of chewing buffer prepared as described above was degassed and heated to 38° C. to prepare a solution (chewing buffer).

900 ml of the obtained chewing buffer was transferred to each vessel in the USP Dissolution Apparatus 1 (e.g. 708-DS Dissolution Apparatus by Agilent). Inside the vessels, the chewing buffer (pH 7.4) was adjusted to 37° C.

Weight measured pouches were transferred to baskets and the dissolution apparatus was started. Sampling times of buffer were set to 2, 6, 12, 20 and 30 min. Rotational speed was set to 100 rpm. Release samples after 2 min were taken manually with cannulas. Release samples after 6, 12, 20 and 30 min were taken automatically with the dissolution apparatus.

All samples were filtered and prepared for HPLC analysis.

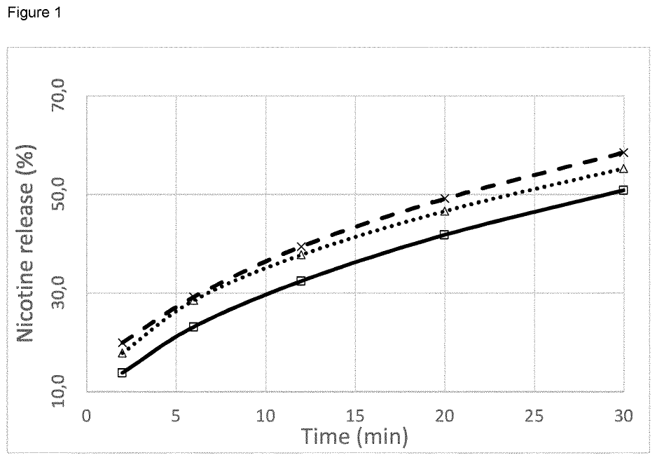

As can be taken from FIG. 1, Example 1 (dotted line) and Example 2 (dashed line), which comprise methyl cellulose as a humectant, exhibit improved (faster) nicotine release compared to Comparative Example 1 (solid line), which comprises sodium alginate as a humectant.

The present invention is also directed to the following embodiments.

Embodiment 1: Use of methyl cellulose as a humectant in a pouch substrate.

Embodiment 2: The use according to embodiment 1, wherein said pouch substrate further comprises one or more carriers and one or more active ingredients.

Embodiment 3: The use according to embodiment 1 or 2, wherein the active ingredient is nicotine.

Embodiment 4: The use according to any one of embodiments 1-3, wherein the methyl cellulose has a viscosity at 20° C. of about 200 to about 10000 cP at 2% w/v concentration.

Embodiment 5: The use according to any one of embodiments 1-4, wherein the methyl cellulose contains about 27.5-31.5 wt % of methoxy groups.

Embodiment 6: The use according to any one of embodiments 1-5, wherein the methyl cellulose has a viscosity at 20° C. of about 300 to about 5000 cP at 2% w/v concentration.

Embodiment 7: The use according to any one of embodiments 1-6, wherein the methyl cellulose is added in an amount of about 0.5 wt %-10 wt % based on the total weight of the pouch substrate.

Embodiment 8: The use according any one of embodiments 1-7, wherein the pouch substrate further comprises one or more flavoring agents.

Embodiment 9: A pouch for oral use comprising a pouch substrate, wherein said pouch substrate comprises one or more carriers, one or more active ingredients and methyl cellulose having a viscosity at 20° C. of about 200 to about 10000 cP at 2% w/v concentration.

Embodiment 10: The pouch for oral use according to embodiment 9, wherein the active ingredient is nicotine.

Embodiment 11: The pouch for oral use according to embodiment 9 or 10, wherein the methyl cellulose contains about 27.5-31.5 wt % of methoxy groups.

Embodiment 12: The pouch for oral use according to any one of embodiments 9-11, wherein the methyl cellulose has a viscosity at 20° C. of about 300 to about 5000 cP at 2% w/v concentration.

Embodiment 13: The pouch for oral use according any one of embodiments 9-12, wherein the amount of methyl cellulose in the pouch substrate is about 0.5 wt %-10 wt % based on the total weight of the pouch substrate.

Embodiment 14: The pouch for oral use according any one of embodiments 9-13, further comprising one or more flavoring agents.

1.-11. (canceled)

12. Methyl cellulose as a humectant in a pouch substrate, wherein the methyl cellulose has a viscosity at 20° C. of about 200 cP to about 10000 cP at 2% w/v concentration, and wherein the methyl cellulose is added in an amount of about 0.5 wt %-6 wt % based on the total weight of the pouch substrate.

13. The methyl cellulose according to claim 11, wherein the pouch substrate further comprises one or more carriers and one or more active ingredients.

14. The methyl cellulose according to claim 12, wherein the one or more active ingredients is nicotine.

15. The methyl cellulose according to claim 12, wherein the methyl cellulose contains about 27.5 wt %-31.5 wt % of methoxy groups.

16. The methyl cellulose according to claim 12, wherein the methyl cellulose has a viscosity at 20° C. of about 300 cP to about 5000 cP at 2% w/v concentration.

17. The methyl cellulose according to claim 12, wherein the pouch substrate further comprises one or more flavoring agents.

18. A pouch for oral use comprising a pouch substrate, wherein the pouch substrate comprising one or more carriers, one or more active ingredients, and methyl cellulose having a viscosity at 20° C. of about 200 cP to about 10000 cP at 2% w/v concentration, wherein the methyl cellulose is added in an amount of about 0.5 wt %-6 wt % based on the total weight of the pouch substrate.

19. The pouch according to claim 18, wherein the active ingredient is nicotine.

20. The pouch according to claim 18, wherein the methyl cellulose contains about 27.5 wt % 31.5 wt % of methoxy groups.

21. The pouch according to claim 18, wherein the methyl cellulose has a viscosity at 20° C. of about 300 cP to about 5000 cP at 2% w/v concentration.

22. The pouch according to claim 18, further comprising one or more flavoring agents.
